(19) United States
(12) Patent Application Publication
(10) Pub. No.: US 2016/0130547 A1
(43) Pub. Date: May 12, 2016

(54) AN APPARATUS FOR CULTURING PHOTOSYNTHETIC ORGANISMS

(71) Applicant: RELIANCE INDUSTRIES LIMITED, Maharashtra (IN)

(72) Inventors: Umesh VENKATARAMU, Navi Mumbai (IN); Vishwanath SAPRE AJIT, Navi Mumbai (IN); Manish BHOLE, Navi Mumbai (IN)

(73) Assignee: RELIANCE INDUSTRIES LIMITED, Mumbai, Maharashtra (IN)

(21) Appl. No.: 14/898,590

(22) PCT Filed: Jun. 13, 2014
(86) PCT No.: PCT/IB2014/062204

§ 371 Date: Dec. 15, 2015

(30) Foreign Application Priority Data
Jun. 17, 2013 (IN) 2037/MUM/2013

(51) Int. Cl. C12M 1/06; C12M 1/36; C12M 1/34; C12M 1/00

(52) U.S. Cl. CPC C12M 27/02; C12M 21/02; C12M 41/48; C12M 27/22; C12M 41/42

(57) ABSTRACT

The present disclosure provides an apparatus for culturing photosynthetic organisms. The apparatus comprising body of liquid of predetermined shape for holding ingredients required for culturing the photosynthetic organisms. The apparatus further comprises a mixing device for mixing ingredients in a body of liquid. The mixing device comprises at least one mixing plate of predetermined shape comprising a plurality of perforations. The mixing plate is adapted to be movably disposed within the body of liquid. Further, at least one drive mechanism coupled to the at least one mixing plate. The drive mechanism is configured to move the at least mixing plate in the body of liquid to accomplish turbulence in the body of liquid for maintaining the photosynthetic organisms in a suspended state to facilitate uniform exposure of photosynthetic organisms to light.

### CROSS-REFERENCE TO RELATED APPLICATIONS

This application is a 371 U.S. National Stage of International Application No. PCT/M2014/062204, filed Jun. 13, 2014, which claims the benefit of and priority to

Indian Patent Application No. 2037/MUM/2013, filed Jun. 17, 2013. The disclosures of the above applications are incorporated herein by reference.

### TECHNICAL FIELD

Embodiments of the present disclosure generally relate to a mixing device and an apparatus for employing the mixing device. The mixing device is adapted to be employed in a body of liquid for mixing of ingredients required for culturing photosynthetic organisms, so as to substantially maintain photosynthetic organisms in a suspended state to facilitate uniform exposure of photosynthetic organisms to light.

### BACKGROUND

With the industrial evolution, different types of bodies of fluid (such as liquid) are increasingly being employed for purposes including, but not limited to, agitation of dispersible and insoluble solid substances, cultivation of organisms (and particularly, photosynthetic microorganisms), fishery, and wastewater treatment. However, solid mass in a body of liquid is required to be kept in a suspended state to avert settling thereof for achieving best results. Specifically, the solid mass is required to be maintained in a suspended state in the body of liquid for achieving one or more objectives, including, but are not limited to, uniform exposure of the solid mass to light, uniform distribution of the solid mass within the body of liquid, and uniform mixing of the solid mass with other ingredients (such as nutrients, other chemical entities etc.) within the body of liquid and the like. To achieve the above one or more objectives, turbulence needs to be generated in the body of liquid to avoid settling of the solid mass.

In conventional practice, the turbulence is generated by providing solid surface substantially flat agitators such as but not limited to a paddlewheel adapted to rotate in a predetermined direction/along an axis including but not limited to a horizontal axis and a semi-horizontal axis; a stirrer adapted to rotate in a predetermined direction/along an axis including but not limiting to a vertical axis and a semi-vertical axis; and a baffle fixed or moving in a predetermined manner including but not limited to a periodic motion and a rotational motion. Typically, all of such conventional solid-surface agitators are either partially or fully submerged in a body of liquid, and are adapted to move or rotate in respective, aforesaid manners for mixing purposes. However, these conventional solid surface agitators, which are substantially flat, require high energy for creating turbulence. Specifically, the conventional agitators with substantially flat solid surface encounter a high resistance provided by various ingredients in the body of liquid, thereby resulting in a high energy requirement for the operation and/or movement thereof for generating turbulence. For large-scale applications it is imperative that the energy consumption in mixing is kept at minimum and yet optimal results are achieved. In addition to the high energy consumption, utilization of conventional agitators may lead to problems including but not limited to, inefficient mixing based on vortices formed behind the agitators and settling of solid mass at areas not within the reach of the agitators that have limited dimensions (such as diameters etc.), cavitation and raising of liners in lined bodies of liquid etc.

The term ‘substantially flat agitator’ as used in this disclosure may be defined as an apparatus which shakes or stirs the components including, but not limited to, water and gases in a body of liquid. The agitators are predominantly two dimensional in shape with a shorter third dimension that is provided for mechanical integrity. Further, the term ‘substantially flat agitator’ may be defined as agitators with flattened surface which may or may not be 100% planar, which may include curved surface or angular surface, and uneven surface, in other words it is not completely planar.

The term ‘body of liquid’ as used herein above and below may relate to a natural body of liquid (such as a natural pond) or man-made body of liquid (such as a pilot/laboratory scale liquid containing vessel which can be a part of any further system) which is independent of dimension and/or shape thereof. Specifically, the body of liquid may comprise components including, but not limiting to, solid mass, liquid and gases. Further, the term ‘solid mass’ may relate to one or more dispersible solid organic/inorganic chemicals, waste materials and recyclable materials; biomass; organisms; ingredients such as nutrients and the like.

As an example, efficient cultivation of photosynthetic organism in a body of liquid such as a reservoir/a pond which is filled with liquid such as water, and nutrients is dependent on exposure of photosynthetic organism to sufficient light, which is necessary for growth of photosynthetic organisms. Traditionally, photosynthetic organisms are grown in raceway ponds filled with water in which turbulences are accomplished by conventional mechanical devices such as agitators including, but not limiting to, paddlewheel and the substantially flat agitators having solid surface. As explained above the conventional agitators encounter higher resistance for operation and/or movement thereof inside the body of liquid, thereby resulting in high energy consumption which in turn increases cost of cultivation of photosynthetic organism.

Limitations of existing conventional substantially flat agitators having solid surfaces such as but not limiting to, paddlewheel, stirrers and baffles are illustrated with the help of cultivation of photosynthetic organism (one of the field of applications of the flat agitators) as an example. However, such example should not be construed as only application. Thus, person skilled in the art can envisage various other applications where such limitation exists.

In light of foregoing discussion, there exists a need to develop a mixing device configured to be employed in bodies of liquid to overcome the limitations as stated above.

### SUMMARY

The shortcomings of the prior art are overcome and additional advantages are provided through the provision of a mixing device and an apparatus as claimed in the present disclosure.

Additional features and advantages are realized through the techniques of the present disclosure. Other embodiments and aspects of the disclosure are described in detail herein and are considered a part of the claimed disclosure.

In one non-limiting embodiment of the present disclosure, there is provided an apparatus for culturing photosynthetic organisms. The apparatus comprising body of liquid of predetermined shape for holding ingredients required for culturing the photosynthetic organisms. The apparatus further comprises a mixing device for mixing components in a body of liquid. The mixing device comprises at least one mixing plate of predetermined shape comprising a plurality of perforations. The mixing plate is adapted to be movably disposed within the body of liquid. Further, at least one drive mechanism is coupled to the at least one mixing plate. The drive mechanism is configured to move the at least mixing plate in the body of liquid to accomplish turbulence in the body of liquid for maintaining the photosynthetic organism in a suspended state facilitate uniform exposure of photosynthetic organisms to light.

In an embodiment of the present disclosure, the perforations are distributed substantially on entire surface of the at least one mixing plate. In alternative embodiment, the perforations are distributed on a portion of surface of the at least one mixing plate.

In an embodiment of the present disclosure, the drive mechanism is at least one of motorized mechanism and actuator mechanism. The drive mechanism moves the at least one mixing plate in at least one of horizontal, vertical, rotational, and angular motion in the body of liquid.

In an embodiment of the present disclosure, the body of liquid is at least one of a manmade body of liquid and a natural body of liquid.

In an embodiment of the present disclosure, the body of liquid comprises a guide rail for moving the drive mechanism. The guide rail is at least one of a rail with substantially flat surface, a cable and a pipe. The mixing device further comprises at least one wheel mounted on the guide rail for moving the drive mechanism.

In an embodiment of the present disclosure, at least one support member is provided for supporting the drive mechanism. The at least one support member comprises at least one guide shaft extending from either sides for contacting side walls of the guide rail. Further, the at least one guide shaft comprises a wheel.

In an embodiment of the present disclosure, the ingredients required for culturing the photosynthetic organisms comprise of water, and nutrients. In an embodiment of the present disclosure, the drive mechanism is powered by at least one of battery, grid power, and photovoltaic panels or combination thereof.

In an embodiment of the present disclosure, the drive mechanism is interfaced with a controller. The controller regulates the direction of movement of the drive mechanism upon reaching preset distance.

The foregoing summary is illustrative only and is not intended to be in any way limiting.

In addition to the illustrative aspects, embodiments, and features described above, further aspects, embodiments, and features will become apparent by reference to the drawings and the following detailed description.

### BRIEF DESCRIPTION OF ACCOMPANYING DRAWINGS

The novel features and characteristic of the disclosure are set forth in the appended claims. The disclosure itself, however, as well as a preferred mode of use, further objectives and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a schematic view of an apparatus for mixing according to an embodiment of the present disclosure.

FIG. 2 illustrates a schematic section of an apparatus for mixing, in accordance with another embodiment of the present disclosure.

FIGS. 3a-3b illustrate schematic views of a mixing device employed in an apparatus used for culturing photosynthetic organisms as an embodiment of the present disclosure.

FIG. 4 illustrates schematic view of a drive mechanism used to move the mixing plate in a body of liquid in accordance with an embodiment of the present disclosure.

FIG. 5 illustrates schematic view of a motorized mechanism used to move the mixing plate in a body of liquid in accordance with an embodiment of the present disclosure.

FIG. 6 illustrates an enlarged view of perforations provided in the mixing plate.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

To overcome the limitations stated in the background, the present disclosure provides an apparatus for culturing of photosynthetic organisms. The apparatus comprises a body of liquid of predetermined shape for holding ingredients required for culturing the photosynthetic organisms. The apparatus also comprises a mixing device hereinafter interchangeably referred to as agitator for mixing the ingredients in the body of liquids, so as to substantially keep the photosynthetic organisms in a suspended state. The mixing device of the present disclosure comprises at least one mixing plate configured to be movably disposed in a body of liquid. The mixing device comprises a plurality of perforations on the at least one mixing plate in order to be configured as an agitator with a porous surface. In one embodiment, the perforations are distributed substantially on entire surface of the mixing plate. In alternative embodiment, the perforations may be distributed on a portion of the surface of the mixing plate. Further, a drive mechanism such as but not limiting to motor mechanism and actuator mechanism is coupled to the mixing plate for moving the mixing plate in the body of liquid to generate the turbulence in the body of liquid to maintain the photosynthetic organisms in the suspended state. Without departing from the scope of the present disclosure, any other mechanism may be employed for moving the mixing plate within the body of liquid.

Further, the body of liquid comprises a guide rail for moving the drive mechanism. In an embodiment of the present disclosure, the guide rail is at least one of a rail with substantially flat surface, a cable and a pipe. In the mixing device, at least one wheel is mounted on the guide rail for moving the drive mechanism. In addition, at least one support member is provided in the body of liquid for supporting the drive mechanism. In an embodiment of the disclosure, the support member may be made of material including but not limiting to wood, ply wood, laminated boards, metal sheets, and polypropylene sheets. The thickness of the support member is configured so as to withstand the weight of the drive mechanism with mixing plate. The support member comprises at least one guide shaft extending from either side for contacting side walls of the guide rail. The guide shafts are provisioned to guide the drive member along with the mixing plate onto the guide rail. In an embodiment, the guide rail may be configured as flat surface/smooth surface. In an embodiment, the wheel can be made of any material such as but not limiting to rubber, nylon, ploy propylene, metal, and any material which is suitable to the surface on which the wheel is expected to roll.

In an embodiment, the drive mechanism may be a motor. Any motor including but not limiting to Direct current motor, and Alternative current motor may be used as driving mechanism. The drive mechanism will be coupled to the wheels for moving the mixing plate in the body of liquid. In an embodiment, the motor will be coupled to the wheels by a power transmission mechanism such as but not limiting to belt drive, chain drive, gear drive, etc. the belt drive may be V-belt drive, and chain drive may be chain and sprocket mechanism. However, any type of belt drive can be used without departing from the scope of the disclosure. In an embodiment of the disclosure, the motor is powered by one or more batteries. The batteries may be rechargeable batteries, and are recharged by the mechanism such as grid power, electric charging, and photovoltaic cells. In an embodiment, the drive mechanism may be operated at different speeds by a controller based on the requirement in the process.

Further, perforations are provided on the mixing plate to accomplish turbulence of the ingredients including, but not limited to, solid mass such as but not limiting to photosynthetic organisms in the body of liquid by allowing the flow of ingredients through the perforations when the mixing plate operates and/or is moved inside the body of liquid. Since, the perforations allow the flow of ingredients in the body of liquid through the mixing plate, less resistance is provided to the operation of the mixing plate as imparted by the ingredients in the body of liquid. This aids in consumption of less power for operating the mixing device. Further, percentage free area of the mixing plate, which is, ratio of the area of perforations on the mixing plate and total cross-sectional area of the mixing plate, may be optimized based on mixing requirement. Specifically, higher the free area, lower is the resistance to the movement of the ingredients in the body of liquid, thereby involving less power consumption for moving the mixing plate. The term free area as used herein above and below relates to area available for liquid movement through the mixing device. Perforation size for a fixed free area may also be optimized for improving mixing quality. Specifically, size of vortices formed behind the mixing device may be determined based on the perforation size for a fixed free area for improving quality of mixing.

Furthermore, due to the provision of the plurality of perforations in the mixing plate, a more complex turbulence of the solid mass can be generated in the body of liquid than the one expected out of a solid block without perforations. Moreover, perforations on the mixing plate also aids in saving energy because of lesser resistance for the operation (liquid being allowed to pass through) of the mixing plate. In addition, the size and arrangement of the perforations are provided in the mixing plate in such a way that it reduces entanglement, agglomeration or adhesion of the dispersible ingredients in and around the perforations and/or on the solid surface.

In addition, the mixing plate is configured to optionally move in a predetermined direction and/or manner including but not limited to, a horizontal translation movement in either a straight line or curvature in at least one axis including, but not limited to, horizontal axis, vertical axis, or any other axis based on the requirements. The movement of the mixing plate within the body of liquid additionally creates a complex turbulence, thereby resulting in mixing of the ingredients including, but not limited to, solid mass in the body of liquid. Due to provision of mixing, the solid particles in the body of liquid remain suspended.

In one non-limiting embodiment of the present disclosure, the turbulence is further generated by planetary motion for the mixing plate in the body of liquid. The term ‘planetary motion’ as used herein above may be defined as essentially having a horizontal, translational motion apart from/irrespective of an axial motion, if any, around the vertical, horizontal or any other axis. In some embodiments, the axial motion can be avoided in order to reduce centrifugal precipitation or agglomeration.

The term mixing plate as used in this disclosure preferably relates to a substantially flat agitator having a porous surface. The substantially flat agitator may not be limited to an agitator with a planar porous surface; a curved porous surface; an uneven porous surface; and an angular porous surface; and like. Further, the agitator may be a porous agitator having a configuration including but not limited to, a paddle wheel, a mixing plate, a baffle, a stirrer and the like. In an embodiment, the mixing plate referred herein above is a plate of predetermined shape such as but not limiting to a rectangular plate and a trapezoidal plate with perforations.

In an embodiment of the present disclosure, the mixing plate may be configured with an aerodynamic structure, and will be provided with perforations. The aerodynamic structure of the mixing plate helps to achieve the movement of the mixing plate in the body of liquid with least resistance for the flow of ingredients, and thereby saves energy.

In an embodiment of the present disclosure, the mixing plate is more conducive to solid dispersants which has a reticular and/or lamellar rather than pure granular disposition.

In an embodiment of the present disclosure, the mixing plate construction may be configured in a shape examples of which include, but are not limited to, a flat shape, a curvature shape, a tapered shape, an angular shape, or any other shape, or combinations thereof, which serves the purpose of the present disclosure. Further, the cross section of the mixing plate is selected from the group comprising but not limiting to at least one of rectangular cross section, circular cross section, square cross section, trapezoidal cross section, triangular cross section, and V-notch cross section. In addition, the perforations provided on the mixing plate are configured in a shape, examples of which include, but are not limited to, a circular shape, a rectangular shape, a triangular shape, a square, hexagonal shape, octagonal shape or any other shape, or combinations thereof, which serve the purpose of the present disclosure. The perforations in mixing plate are configured such that the ingredients will flow through the perforation without imparting resistance on the mixing plate. For example, the average opening of perforation is in range of 68-76% of the total outer measurement of mixing plate.

Further, the mixing plate may be made up of a light-weight material such as a plastic material and a fiber reinforced plastic (FRP) material, which requires minimum power for operation and/or movement thereof. In another embodiment, the mixing plate may be configured in the form of a mesh or a net made of wires composed of materials such as a metallic material, a plastic material, a polymeric material, a combination thereof, and the like, for mixing. For example, the mixing plate may be configured in the form of a net made of High-density polyethylene (HDPE), Netlon, Nylon Aluminum, and stainless steel material. In yet another embodiment, the mixing plate may be configured as either a perforated metal or a polymeric sheet. The aforesaid materials or configurations of the mixing plate are only for exemplary purposes and need not be considered to be limiting to the scope of the present disclosure.

In an embodiment of the present disclosure, the mixing plate is supported by a support frame for mounting onto the drive mechanism. The support frame may be at least one of but not limiting to metal sections, rods, and tubes. The support frame is fixed to peripheral edges of the mixing plate to enclose and carry the mixing plate. In an embodiment, the support frame may be made of material including but not limiting to metal, synthetic polymeric material, and combination thereof.

For better mixing, speed of motion of the mixing plate may be optimized. Specifically, higher speed of motion for the mixing device ensures better mixing. The time lag between two successive visits of the mixing device at a same location in the body of liquid may be optimized/designed based on the dimensions of the body of liquid, speed of motion of the mixing device and the number of mixing devices installed along a channel length/width of the body of liquid.

In an embodiment of the present disclosure, the body of liquid may be at least one of natural body of liquid and manmade body of liquid. In an embodiment, the body of liquid may be configured in any shape including but not limiting to rectangular cross section, V-notch cross section, circular cross section and square cross section.

Without departing from the scope of the present disclosure, the body of liquid may be of any shape and dimension as required for the purposes of the present disclosure. Further, the body of liquid may be made of materials such as cement, metals, glass, plastic, any other masonry material and the like, which are known in the art, and with or without a liner material.

Henceforth, the present disclosure is explained with the help of one or more exemplary embodiments in conjunction with the drawings. However such exemplary embodiments should not be construed as limitations of the present disclosure. The person skilled in the art can envisage various such embodiments without deviating from scope of the present disclosure.

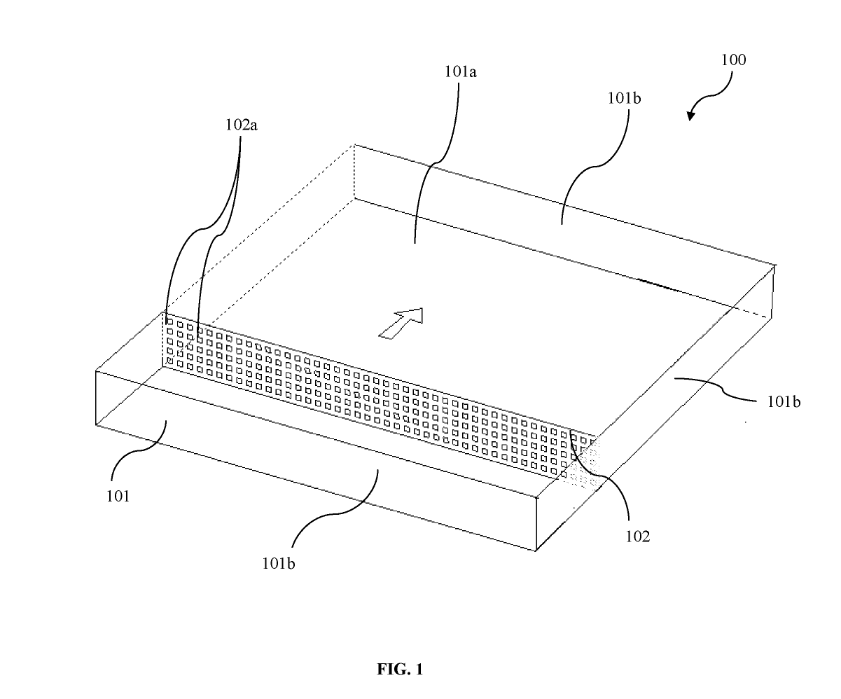

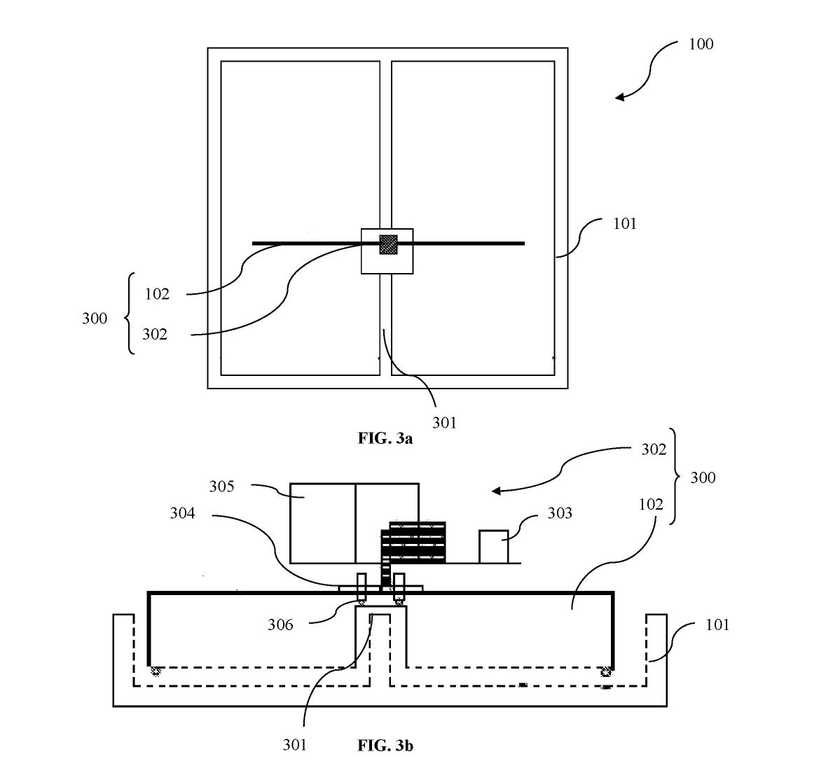

FIG. 1 is an exemplary embodiment of the present disclosure which illustrates a schematic view of an apparatus (100) employed for mixing. The apparatus (100) comprises a body of liquid (101) which is either a natural body of liquid (such as a natural pond) or man-made body of liquid (such as a pilot/laboratory scale liquid containing vessel which can be a part of any further system). The body of liquid (101) may contain ingredients such as liquids, solid mass, and optionally gaseous particles. In an embodiment of the present disclosure, the body of liquid (101) may also be configured to hold fluids. The body of liquid (101) as shown in FIG. 1 is of rectangular shape, and may be configured as at least one of culture medium, fish rearing medium, and waste water treatment medium. One or more mixing plates (102) such as flat agitator having plurality of perforations of predetermined shape are movably disposed in the body of liquid (101). Further, the mixing plate (102) is driven by a drive mechanism [not shown] such as but not limiting to motorized mechanism and an actuator mechanism is provided in the body of liquid (101). The mixing plate (102) is configured with the drive mechanism to form a mixing device (300) in conjunction with FIGS. 1-6. The one or more mixing plate (102) is either removably or non-removably fitted to the drive mechanism [as shown in FIGS. 3a-b], within the body of liquid (101) with sufficient clearance from a bottom surface (101a) and side walls (101b) of the body of liquid (101). The body of liquid (101) comprises a guide rail (not shown) for guiding the mixing plate (102) in the body of liquid (101). The mixing plate (102) may move in predetermined direction and make horizontal translational movement in either straight line along length or width of the body of liquid (101) or travels in curvature within the body of liquid (101). Thus, the movement of the mixing plate (102) helps in mixing the ingredients in the body of liquid (101) to facilitate uniform distribution of the solid mass within the body of liquid (101), and to maintain the solid mass such as but not limiting to photosynthetic organisms in suspended state within the body of liquid. Further, the mixing plate (102) is provided with a plurality of perforations (102a) for efficient mixing. In one embodiment the perforations (102a) are distributed substantially on entire surface of the mixing plate (102). When the mixing plate (102) travels in the body of liquid (101), turbulence is generated due to perforations (102a) provided on the mixing plate (102), which results in surface renewal of the solid mass.

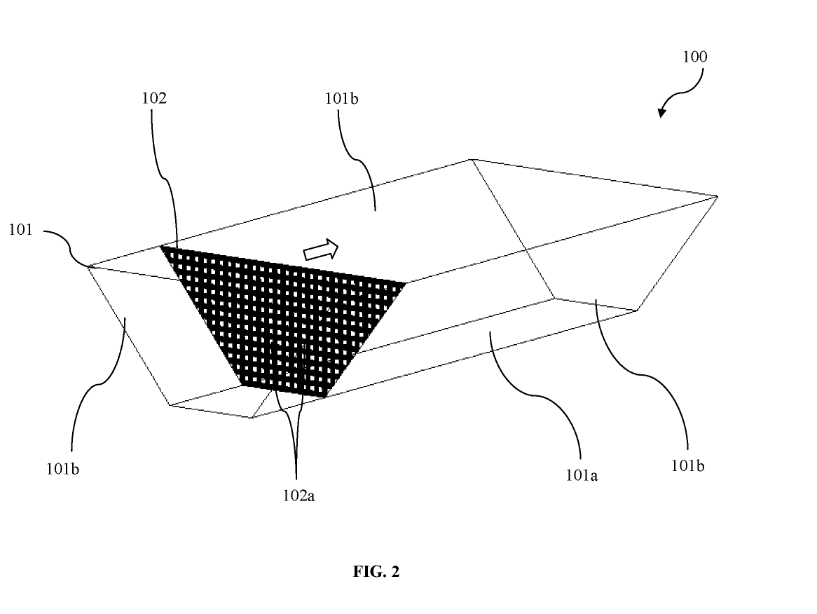

FIG. 2 is another exemplary embodiment of the present disclosure which illustrates a schematic view of an apparatus (100) and a V-notch shaped body of liquid (101) employing at least one mixing plate (102). The apparatus (100) includes the body of liquid (101) having a V-notch shaped design for minimizing the energy consumption in mixing ingredients in the body of liquid (101). One or more mixing plate (102) of predetermined shape such as but not limiting to V-notch shape, circular shape, rectangular shape, square shape are movably disposed in the body of liquid (100) for mixing the ingredients in the body of liquid (101). For the purpose of this description, the mixing plate (102) is configured as a V-notch shaped plate. Further, the mixing plate (102) is driven by a drive mechanism [not shown] such as, but not limiting to, motorized mechanism and actuator mechanism provided in the body of liquid (101). The mixing plate (102) is configured with the drive mechanism to form a mixing device (300) [best shown in FIGS. 3a and 3b]. The mixing plate (102) is either removably or non removably fitted to a drive mechanism in the body of liquid (101) with sufficient clearance from the bottom surface (101a) and the side walls (101b) of the body of liquid (101). The mixing plate (102) moves in a predetermined direction and makes horizontal translational movement along length or width of the body of liquid (101) or travels in a curvature in the body of liquid (101). Without limitation to the scope of the disclosure, the mixing plate (102) may make a non-translational movement within the body of liquid (101).

The movement of the mixing plate (102) enables mixing of ingredients in the body of liquid (101) to facilitate uniform distribution of the solid mass within the body of liquid (101), and to maintain the solid mass such as but not limiting to photosynthetic organisms in suspended state within the body of liquid (101). Further, the mixing plate (102) is provided with a plurality of perforations (102a). In one embodiment, the perforations (102a) are distributed substantially on entire surface of the mixing plate (102). In yet another embodiment, the perforations (102a) may be distributed on a portion of the surface of the mixing plate (102). When, the mixing plate (102) travels in the body of liquid (101), turbulence is generated due to presence of perforations (102a) on the mixing plate (102), which results in surface renewal of the solid mass. In an embodiment of the present disclosure, the number of mixing plate (102), speed of motion of the mixing plate (102), and porosity of the mixing plate (102) can be optimized using flow modeling and mixing analysis.

As an example of the present disclosure, the apparatus comprising “V-notch” shaped design of body of liquid (101) is employed with the at least one mixing device (300) may be used for cultivation of organisms such as photosynthetic organisms. The culturing apparatus (100) includes a body of liquid (101) having a V-notch shaped design for minimizing the energy consumption in mixing ingredients in the body of liquid (101) for photosynthetic organisms cultivation. The mixing device (300) comprising one or more mixing plate (102) of V-notch shape confirming to the shape of the body of liquid (101) is movably disposed, and is either removably or non removably fitted to a drive mechanism in the body of liquid (101). The mixing plate (102) moves in a predetermined direction and makes horizontal translational movement along length or width of the body of liquid (101) or travels in a curvature in the body of liquid (101). Without limitation to the scope of the disclosure, the mixing device (102) may make a non-translational movement within the body of liquid (101). The movement of the mixing device (102) enables mixing the photosynthetic organisms in growth medium including but not limited to water and nutrients in the body of liquid (101) to facilitate periodic uniform exposure of photosynthetic organisms to light, uniform distribution of the photosynthetic organisms within the body of liquid (101), and uniform mixing of the photosynthetic organisms with other ingredients (such as nutrients, other chemical entities etc.) within the body of liquid (101).

The following aspects are envisioned for V-notch shaped design of the photosynthetic organisms' cultivation apparatus as advantageous over traditional raceway pond.

In one aspect, the apparatus (100) for photosynthetic organisms' culturing having V-notch cross-section has a smaller width compared to traditional raceway design for same amount of volume. Since the mixing energy consumption is largely decided by width of the body of liquid, the apparatus (100) having V-notch shape would incur less energy consumption compared to traditional raceway pond. Further, the mixing plate (102) in translational (linear) motion creates vigorous turbulence in its wake promoting a greater localized mixing compared to poor global mixing in a traditional raceway design.

In another aspect, amount of liner material required for the apparatus (100) for photosynthetic organisms culturing having V-notch geometry would be lesser than that of a standard rectangular design. The potential liner material savings depends upon the specification of V-notch width and depth. It must be noted that the liner savings are compared for the same wetted-volume of rectangular design.

In further aspect, V-notch cross-section design of the body of liquid (101) offers a unique advantage for cleaning the body of liquid (101). The precipitates, dead-photosynthetic organisms biomass would naturally accumulate on the floor/bottom surface (101a) and can be cleaned very easily compared to a rectangular design. This reduces the cost, since the cleaning of the body of liquid (101) is a very labor-intensive process.

In furthermore aspect, the depth of water in body of liquid (101) having V-notch shape is higher than that of the body of liquid (101) having rectangular shape. Thus, the body of liquid (101) having V-notch shape would save land area as compared to the body of liquid (101) having rectangular shape. Therefore, body of liquid (101) having V-notch shape would be especially useful where space is a critical issue. Further, therefore as opposed to a conventional paddle wheel type of mixing device, a V-notch shaped mixing plate (102) adapted in the V-notch shaped boy of liquid (101) for increasing mixing efficiency.

In one embodiment, the body of liquid (101) having V-notch shape can be a V-shaped photo-bioreactor or any another closed system where such requirement of mixing exists.

As an example, a raceway type body of liquid (101), such as an open raceway pond, having a rectangular design (as shown in FIG. 1) which may hold a volume of water/culture of about 200 KL, with an area of about 1000 sq·m and depth of about 20 cm; the area of the pond liner required (including partition wall and anchoring around the pond, free board above water level etc.) is about 1225 sq·m for a pond of 100 m×10 m size. However, in V-notch design of the body of liquid (101) with volume of water/culture of about 200 KL, area of about 500 sq·m (exposed to sun), depth of culture of about 80 cm; the area of liner required (including anchoring around the pond, free board above water level etc.) is about 598 sq·m. Thus, it may be seen for handling same liquid volume the V-notch configuration requires less land area (by 50%) and lesser Liner area (50%) and only the mixing device may provide effective mixing for such configuration.

The following description of the present disclosure iterates the apparatus having a body of liquid and at least one mixing device employed in the body of liquid. The FIGS. 3a and 3b are exemplary embodiment of the present disclosure which illustrates top view and front view of an apparatus (100) employing the mixing device (300). The apparatus (100) may be used for applications including but not liming to culturing of photosynthetic organisms such as but not limiting to algae, fish rearing and waste water treatment. The mixing device (300) may be embedded in a body of liquid (101) of predetermined shape for holding ingredients such as but not limiting to liquids, solid mass, and optionally along with gaseous molecules in a suspended state. The body of liquid (101) may be either natural body of liquid or manmade body of liquid. Further, the apparatus (100) includes mixing device (300) for mixing the ingredients in the body of liquid so as to maintain the solid mass in a substantially the suspended state. The mixing device (300) includes one or more mixing plates (102) with a plurality of perforations (102a). Further, a drive mechanism (302) is provided in the mixing device (300), wherein, the drive mechanism (302) is coupled to the mixing plate (102) for moving the mixing plate (102) in the body of liquid (101) to generate the turbulence in the body of liquid (101). As shown in FIG. 3a, within the body of liquid (101) there is provided at least one guide rail (301) for moving the drive mechanism in the body of liquid (101). When the drive mechanism (302) is moved on the guide rail (301), it carries the mixing plate (102) coupled to it and thereby generates turbulence in the body of liquid (101). This helps to achieve uniform mixture of ingredients in the body of liquid (101), and also helps to maintain the solid mass in the suspended state.

The drive mechanism (302) is configured to move the mixing plate (102) in a predetermined direction, and makes horizontal translational movement along length or width of the body of liquid (101) or travels in a curvature in the body of liquid (101). Without limitation to the scope of the disclosure, the mixing plate (102) may make a non-translational movement within the body of liquid (101).

In an embodiment of the present disclosure, the apparatus (100) comprises a controller (303) for closed loop operation of the drive mechanism (302). The controller (303) is interfaced with one more limit switches (not shown) for limiting movement of the mixing device (300) in the body of liquid (101). In an embodiment of the present disclosure, the type of limit switches include but are not limited to mechanical-contact type and electronic-non contact type. The limit switches are provided in desired location of the body of liquid (101) so as to limit the movement of the mixing plate (102). Alternatively, a controller may also be provided at a location of a user for operating the drive mechanism (302).

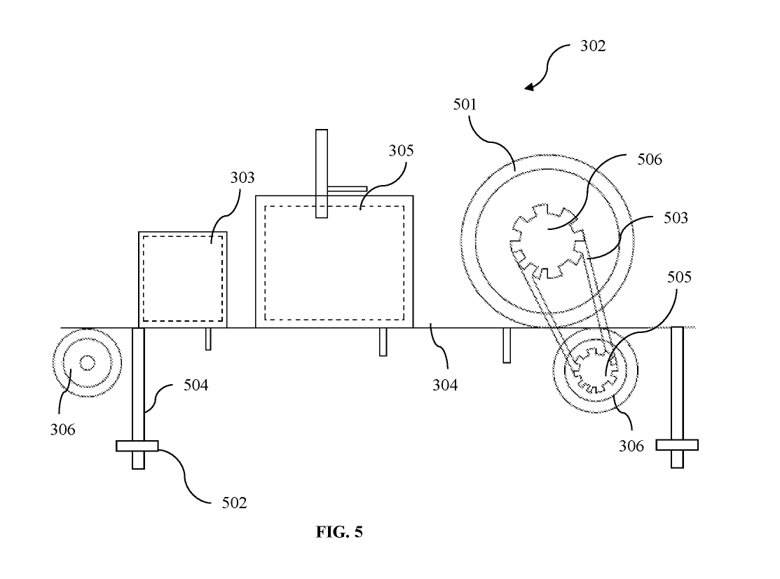

In an embodiment of the present disclosure, the guide rail (301) is at least one of a rail with substantially flat surface, a cable and a pipe. In the apparatus (100), at least one wheel (306) is mounted on the guide rail (301) for moving the drive mechanism (302). In addition, at least one support member (304) is provided in the apparatus (100) for supporting the drive mechanism (302). In an embodiment of the disclosure, the support member (304) may be made of material including but not limiting to wood, ply wood, laminated boards, metal sheets, and polypropylene sheets. The thickness of the support member (304) is configured so as to withstand the weight of the drive mechanism (302) with the mixing plate (102). The support member (304) comprises at least one support/guide shaft (504) [more clearly shown in FIG. 5] extending from either sides for contacting side walls of the guide rail (301). The guide/support shafts (504) are provisioned to guide the drive mechanism (302) along with the mixing plate (102) onto the guide rail (301). In an embodiment, the guide rail (301) may be configured as flat surface/smooth surface. In an embodiment, the wheel (306) can be made of any material such as but not limiting to rubber, nylon, ploy propylene, metal, and any material which is suitable to the surface on which it is expected to roll. In an embodiment, an enclosure (not shown) made of suitable material such as metal, plastic, and composite material is provided in the apparatus (100) for enclosing the drive mechanism (302).

Further, the drive mechanism (302) is interfaced with the controller (303), and said controller (303) is configured to control the speed of the drive mechanism (302) which in turn controls speed of the mixing plate (102). The controller (303) is also configured to control the resting period of drive mechanism (302) between the different travel cycles so as to reduce the power consumption, and to improve the efficiency of mixing of ingredients in the body of liquid (101). In an embodiment of the present disclosure, the controller (303) is configured to either automatically or manually regulate the direction of travel of the drive mechanism (302). This will help the drive mechanism (302) to automatically or manually reverse the direction of travel in the body of liquid (101) after travelling to predetermined distance. For example, the controller (303) regulates the drive mechanism to move in forward direction till it reaches one end of the body of liquid (101), and after reaching the end, the controller (303) regulates the drive mechanism (302) to move in reverse direction till it reaches other end of the body of liquid (101).

In the body of liquid (101), the guide rail (301) is provided in a predetermined location such as at the center of the body of liquid (101) and along a width thereof, or at the ends of the body of liquid (101), or any desired location, and a drive mechanism (302) will be provided on the guide rail (301) to move one or more mixing plate (102) which are either removably or non-removably fitted to the drive mechanism (302), as depicted in FIG. 3b.

The guide rail (301) may be configured over a mid-wall in the body of liquid (101) or guide rail (301) may serve as the mid-wall of the body of liquid (101). In the FIGS. 3a and 3b the mixing plate (102) are shown without any perforations for the purpose of simplicity. However, it should be understood that the mixing plate (102) shown in FIG. 3b is similar to the mixing plate (102) of FIGS. 1 and 2, and may include appropriate number, size, shape and type of perforations as described earlier.

In an embodiment of the present disclosure, the guide rail (301) is provided substantially at the center of the body of liquid (101), in an embodiment on the mid-wall of the body of liquid (101), and includes at least one of but not limited to a rail, pipe, cable and track.

The drive mechanism (302) is provided on the guide rail (301) for movement thereof across the body of liquid (101).

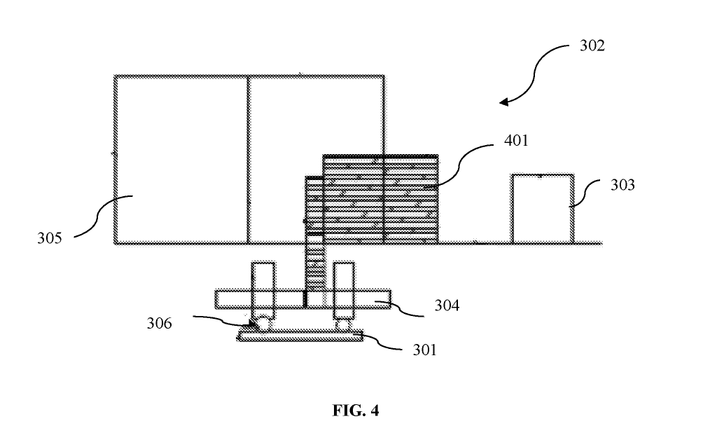

FIG. 4 depict schematic view of an exemplary embodiment of the present disclosure which illustrates a drive mechanism (302) used to move one or more mixing plate (102) in a body of liquid (101). The drive mechanism (302) comprises drive member (401) such as but not limiting to motor and the actuator for moving the mixing plate (102). The drive member (401) is powered by a battery or power source. Further, one or more wheels (306) are provided in the drive mechanism (302) for moving the drive mechanism (302) on the guide rail (301). Furthermore, at least one support member (304) is provided in between the drive mechanism (302) and the guide rail (301), to support the drive mechanism (302), and is configured to move on the guide rail (301) through the one or more wheels (306). Without limiting the scope of the present disclosure, the one or more wheels (306) may be configured to move over the guide rail (301) based on the configuration of the supporting member (304). In one embodiment the support member (304) is further supported on the guide rail (301) with one or more support wheels (not shown) joined to the support member (304) through a support shaft (504) [best shown in FIG. 5] for moving the mixing plate (102) over the guide rails (301).

In an embodiment of the present disclosure, the drive mechanism (302) is operated by the one or more rechargeable batteries (not shown). The batteries are configured to be recharged by at least one mode such as but not limiting to current source, grid power source, and photovoltaic cell source. The batteries are interfaced with the controller (303), and said controller (303) is configured to regulate the charging of the batteries. A cut-off switch (not shown) including but not limiting to relay and MOSFET is provided to automatically regulate the charging. Further, the drive mechanism (302) may be provided with a volt meter to monitor voltage of the battery.

In an exemplary embodiment of present disclosure, the drive mechanism (302) is a motorized mechanism as shown in FIG. 5. The motorized mechanism includes at least one motor (501) which is mounted on the support member (304), and is connected to the one or more wheels (306) for moving the support member (304). In an embodiment of the present disclosure, the motor (501) is an electric motor, which is either direct current motor or an alternative current motor. In one embodiment, the at least one motor (501) is coupled to the one or more support wheels (502). The at least one motor (501) is coupled using at least one of a chain drive (503), gear drive, and a belt drive [not shown]. For the use of the chain drive (503), a driven sprocket (505) is provided on the one or more wheels (306) and a driving sprocket (506) is provided on the electric motor (501), and a chain is coupled between the driven sprocket (505) and the driving sprocket (506). In an embodiment, the chain/belt may be “V-belt”. The motorized mechanism further comprises a battery (305) interfaced with the electric motor (501) for providing necessary power to the electric motor (501) to move the one or more wheels (306) and/or the one or more support wheels (502). In an embodiment of the present disclosure, a pair of mixing plate (102) are connected to either ends of the support member (304), and the support member (304) carries the pair of mixing plate (102) to mix the ingredients including, but not limited to, solid mass such as but not limiting to photosynthetic organisms in the body of liquid (101) when the motorized mechanism is actuated. The controller (303) may be operated from site of the body of liquid (101) or from a remote location.

In an alternate embodiment of the present disclosure, the mixing plate (102) is moved on the guide rail (301) inside the body of liquid (101) by a mechanism including, but not limited to, rack and pinion mechanism, a hydraulic actuator mechanism, a pneumatic actuator mechanism, or any other mechanism which serves the purpose without going beyond the scope of the present disclosure.

In an exemplary embodiment one can envisage having more than one guide rail (301) to facilitate movement of the mixing plate (102). This could be either to carry more weight of mixing plate (102) or to provide multiple paths/directions for the mixing plate (102) to move.

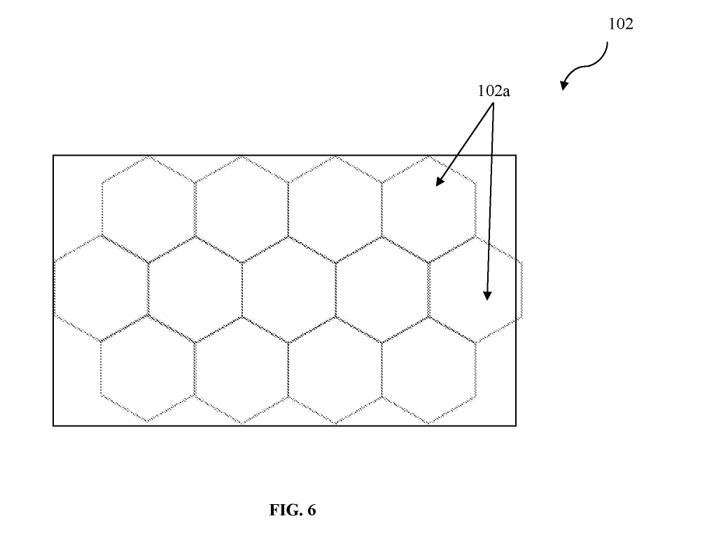

FIG. 6 is an exemplary embodiment of the present disclosure which illustrates enlarged view of perforations (102a) provided in the mixing plate (102). The perforations provided on the mixing plate (102) are configured in a shape examples of which include, but are not limited to a circular shape, a rectangular shape, a triangular shape, a square, hexagonal shape, semi hexagonal shape, octagonal shape or any other shape, or combinations thereof, which serve the purpose of the present disclosure. The perforations (102a) in mixing plate are configured such that the ingredients in the body of liquid (101) will flow through the perforations (102a) imparting least resistance on the mixing plate (102). For example, the average opening of the perforations is in range of 68-76%, preferably 72% of the total outer measurement of mixing plate.

### Advantages:

The present disclosure provides a mixing device for mixing the ingredients in the body of liquid which helps to keep the solid mass in the body of liquid in the suspended state. Thereby, improves the efficiency of the process.

The present disclosure provides a mixing device for mixing the ingredients in the body of liquid in which the volume of mixing can be increased several folds by increasing the running length of the mixing device without increasing the motor power. Thus scale up benefit can be extended by several folds.

The present disclosure provides a mixing device for mixing the ingredients in the body of liquid which has perforations in a mixing plate thereof. This consumes less energy for its operation due to less resistance for the flow of ingredients through the mixing plate.

### Exemplary Experimental Data:

The comparative study has been conducted between conventional mixing devices such as paddle wheels having flat agitators, and a mixing device of the present disclosure. For comparative study, cultivation of photosynthetic organism such as algae is considered.

### Field Data With Regard to Paddle Wheel-Conventional Mixing System in Algae Cultivation:

The data collected is from a 500 sq·m pond.
The width of the paddle wheel is 4.80 metres.
The water depth, on an average, is maintained between 15 cm and 20 cm.
Drive A.C. geared motor of 415 V, 1.5 KW is used.
The velocity of the water flow created is between 30 to 35 cm/sec depending on the depth.
The measured current during a stable operation is 2.7 amperes (76% of motor's rated full load current of Amperes).
The absorbed energy is 1.732×2.7×440×0.8 (PF)=1646 watt hour/hr.

Field data for mixing device having mixing plate with perforations an alternate to paddle wheels:
The data collected is from 500 sq m pond.
The width of the blade that pushes the water in the channel is 4.7 meters.
The speed of the mixing device is about 45 cm/sec and running length is 32 meters.
Time taken to cover 32 metres is 78 sec equivalent to 41 cm per sec.
The mixing device trolley is driven by 48 Volt bi directional D.C. motor.
The motor is energized by a set of four, 12 V, batteries connected in parallel.
The battery is rechargeable through A.C. Socket and also through Photovoltaic Panels mounted on the mixing device trolley.
The measured current during stable running is 1.5 to 1.7 amps @ 48 V.
The absorbed energy, then 48×1.7=81.6 watt hour/hr.

### Results:

The velocity of the mixing device of the present disclosure was matched to that of water flow created by paddle wheel. The physical turbulence mark of mixing is considered to be better than paddle wheel generated flow due to the bottom-top mixing by the presence of perforated screen.

The mixing device of the present disclosure saves 95% of energy cost incurred when compared to paddle wheel operation in view of the above exemplary comparative data-A typical commercial unit in Algae culture like Dunaliella or Spirulina employs raceway ponds as big as 3000 m² and paddle wheels for mixing at 3.75 kwh/hour. Such production systems run about 40 to 100 raceway ponds at any given time. For a 20 hour operation in a day, this amounts to about 3000 kwh/day in a 40 pond operation. Replacing these with mixing device of the present disclosure, it is likely to save 2850 kwh/day.

### Equivalents:

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1-17. (canceled)

18. An apparatus for culturing photosynthetic organisms, the apparatus comprising:
a body of liquid of predetermined shape for holding ingredients required for culturing the photosynthetic organisms; and
a mixing device for mixing ingredients in a body of liquid, said mixing device comprises:
at least one mixing plate of predetermined shape comprising a plurality of perforations, wherein, the at least one mixing plate is adapted to be movably disposed within the body of liquid; and
at least one drive mechanism coupled to the at least one mixing plate, wherein the drive mechanism is configured to move the at least mixing plate in the body of liquid to accomplish turbulence in the body of liquid for maintaining the photosynthetic organisms in a suspended state, to facilitate uniform exposure of photosynthetic organisms to light.

19. The apparatus as claimed in claim 18, wherein the perforations are distributed substantially on entire surface of the at least one mixing plate.

20. The apparatus as claimed in claim 18, wherein the perforations are distributed on a portion of surface of the at least one mixing plate.

21. The apparatus as claimed in claim 18, wherein the drive mechanism is at least one of motorized mechanism and actuator mechanism.

22. The apparatus as claimed in claim 18, wherein the drive mechanism moves the at least one mixing plate in at least one of horizontal, vertical, rotational, and angular motion in the body of liquid.

23. The apparatus as claimed in claim 18, wherein the body of liquid is at least one of a manmade body of liquid and a natural body of liquid.

24. The apparatus as claimed in claim 18, wherein the body of liquid comprises a guide rail for moving the drive mechanism.

25. The apparatus as claimed in claim 24, wherein the guide rail is at least one of a rail with substantially flat surface, a cable and a pipe.

26. The apparatus as claimed in claim 24 comprises at least one wheel mounted on the guide rail for moving the drive mechanism.

27. The apparatus as claimed in claim 18 comprises at least one support member for supporting the drive mechanism.

28. The apparatus as claimed in claim 27, wherein the at least one support member comprises at least one guide shaft extending from either sides.

29. The apparatus as claimed in claim 28, wherein the at least one guide shaft comprises a wheel.

30. The apparatus as claimed in claim 18, wherein the ingredients required for culturing the photosynthetic organisms comprise of water, and nutrients.

31. The apparatus as claimed in claim 18, wherein the drive mechanism is powered by at least one of battery, grid power, and photovoltaic panels or a combination thereof.

32. The apparatus as claimed in claim 18, wherein the drive mechanism is interfaced with a controller.

33. The apparatus as claimed in claim 32, wherein the controller regulates the direction of movement of the drive mechanism upon reaching a preset distance.
